(19) United States
(12) Patent Application Publication
(10) Pub. No.: US 2002/0031535 A1
(43) Pub. Date: Mar. 14, 2002

(54) Topical formulation to treat skin disorders

(76) Inventor: Felix H. Sheffield SR., Colquitt, GA (US)

Correspondence Address: Brian D. Bellamy, P.O. Box 1997, Thomasville, GA, 31799-1997, US

(21) Appl. No.: 09/950,150

(22) Filed: Sep. 10, 2001

Related U.S. Application Data
(60) Provisional application No. 60/232,116, filed on Sep. 11, 2000.

(51) Int. Cl. A61K 7/00

(52) U.S. Cl. 424/401

(57) ABSTRACT

A topical formulation to treat skin conditions associated with skin cancer or other surface oriented skin disorders. The topical formulation includes the ingredients of copus or copper, bluestone, soda, turpentine, alcohol, and water. The topical formulation removes is applied to the affected area of skin to remove the associated skin disfigurement.

This application claims the benefit of U.S. Provisional Application No. 60/232,116, filed Sep. 11, 2000.

The present invention relates generally to the treatment of certain types of skin disorders. More particularly, the topical formulation is used to treat skin conditions associated with skin cancer, including melanoma, basal cell carcinoma, and squamous cell carcinoma, and/or used to treat other surface oriented skin disorders.

It is well known that skin cancer may be removed by one of several methods including excisional surgery, curettage-electrodesiccation, cryosurgery, radiation therapy, topical chemotherapy, or by Mohs micrographic surgery. What is still needed is a topical formulation for removal of surface oriented skin cancer and other skin disorders.

The essential ingredients used to treat the abovementioned conditions comprise: copus or copper, bluestone, soda, turpentine, alcohol and water. In the preferred embodiment, the formula will include these ingredients in the following amounts:

2 tablespoons of the selection of copus or copper;

2 tablespoons of bluestone;

1 tablespoon of soda;

2 tablespoons of turpentine;

1 cup of alcohol;

1 cup of water.

The method by which the topical formulation is made comprises the steps of:

1. Mixing soda, turpentine and alcohol together to form a mixture;

2. Bringing water to a boil;

2. Dissolving bluestone and a selection of copus or copper in the hot boiling water to form a solution;

3. Pouring the solution of bluestone and copus or copper into the mixture of soda, turpentine and alcohol.

4. Stirring the combination of ingredients for about 30 minutes until well-combined.

5. Placing the final product into a suitable container.

The topical formulation is applied to the affected area of skin twice daily until the undesirable skin condition is removed.

I claim:

1. A topical formulation to treat skin disorders consisting essentially of copus, bluestone, soda, turpentine, alcohol, and water.

2. A topical formulation to treat skin disorders consisting essentially of copper, bluestone, soda, turpentine, alcohol, and water.
